(19) United States
(12) Patent Application Publication
(10) Pub. No.: US 2026/0207432 A1
(43) Pub. Date: Jul. 23, 2026

(54) MEDICAL SYRINGE WITH FILTERING NEEDLE CAP

(71) Applicant: Becton, Dickinson and Company, Franklin Lakes, NJ (US)

(72) Inventors: Praveen Nalawade, Belgaum, Karnataka (IN); Shishir Prasad, Ramsey, NJ (US); Manish Kumar, Belgaluru, Karnataka (IN)

(73) Assignee: Becton, Dickinson and Company, Franklin Lakes, NJ (US)

(21) Appl. No.: 19/137,996

(22) PCT Filed: Apr. 27, 2023
(86) PCT No.: PCT/US2023/020134

§ 371 Date: Jun. 11, 2025

Related U.S. Application Data
(63) Continuation-in-part of application No. 18/080,428, filed on Dec. 13, 2022.

(51) Int. Cl. A61J 1/20; A61M 5/178; A61M 5/31; A61M 5/32; A61M 5/34

(52) U.S. Cl. CPC A61J 1/2079; A61M 5/1782; A61M 5/3145; A61M 5/3202; A61M 5/343; A61M 5/347; A61J 1/2096; A61M 2005/3114; A61M 2005/312

(57) ABSTRACT

An open tip, tapered profile, needle cap for a medical syringe facilitates aspiration of filtered medicine into the syringe, through the septum of a medicine vial. A needle filter within a cavity of the needle cap has a distal end facing the open cap tip and a proximal end in abutting, fluid-sealed contact with the needle tip. The needle filter captures particulate matter in medicine that is aspirated through the hollow needle into the syringe cavity while inhibiting fluid flow therethrough external the hollow needle.

### TECHNICAL FIELD

The present disclosure generally relates to a medical injection syringe with a needle cap having a filter. More particularly, embodiments of the syringe with needle cap of the present disclosure facilitates aspiration of medicine from a vial through its septum; medicine is filtered through a needle filter within the needle cap and thereafter drawn into an injection needle of the syringe, while the needle cap is coupled to the needle hub of the syringe.

### BACKGROUND

Liquid drugs and other pharmaceuticals are typically stored in glass ampules or vials and sealed with elastomer stoppers, including by way of non-limiting example stoppers with slit septums. Typically, a two-stage drug administration process is employed to aspirate the drug contained within the vial to a syringe with a fill needle that is subsequently replaced with a hypodermic injection needle prior to administration to a patient. In this typical two-stage process, a cylindrical exterior profile, blunt fill needle is affixed to a Luer connector of the syringe, in order to pierce the stopper/septum, followed by retraction of the syringe plunger to aspirate the drug into the syringe chamber. After filling the syringe with a desired quantity of the drug, the blunt fill needle is then detached from the syringe and replaced with a hypodermic injection needle for injection of the drug into the patient.

In the case of glass ampule-type medicine vials, in order to gain access to the pharmaceuticals, the ampule is opened by snapping the glass neck. Glass neck snapping may introduce glass shards debris in the pharmaceutical, contaminating the drug payload. The shards must be removed from the contaminated pharmaceutical prior to administration to a patient. The glass shards or other debris are typically removed by drawing up the contaminated pharmaceutical through a filtering device secured to the end of the syringe. The filtering device typically comprises a fill needle, cannula or straw that incorporates a filter element.

In the two-stage filtering and drug administration process, a needle or straw has a filter element secured in the needle and connector hub portion. As the liquid payload is drawn up into the syringe, the filter traps the debris, removing it from the liquid payload to be administered. The filtering straw or needle is then removed from the syringe and discarded. A replacement, hypodermic injection needle is then affixed to the syringe for administration of the medicine contained within the syringe to the patient. In order to avoid inadvertent administration of contaminated liquid payload to the patient, care must be taken to remove and discard the filtered needle and replace it with an injection-administration needle/hub.

Attachment and replacement of two separate types of fill and injection needles to the syringe is cumbersome and time consuming. Each separate attachment introduces potential drug administration errors and/or contamination. When a blunt fill needle (whether filtered or non-filtered) is separated from the syringe, any remaining drug aspirated into the fill needle internal cavity that is not contained within the syringe cavity is wasted. Generally cylindrical profile blunt fill needles do not allow complete aspiration of all of the drug contained within the vial because the flat needle tip cannot remain in contact with residual drug contained within the bottom corners of the vial.

There are blunt, cylindrical profile needle caps with filter elements that attach to hypodermic injection needles of affixed syringes. During syringe aspiration, medicine is drawn into an open distal tip of the filtering cap. Filtered medicine flows through the attached injection needle into the syringe barrel. The blunt, filtered needle cap wastes any remaining drug aspirated into “dead space” of the cap as well as leaving drug within the corners of the vial that cannot be drawn through the cap's blunt tip.

### SUMMARY

A medical syringe with an open tip, tapered profile, needle cap facilitates aspiration of filtered medicine into the syringe, through the septum of a medicine vial. A needle filter within a cavity of the needle cap has a distal end facing the open cap tip and a proximal end in abutting, fluid-sealed contact with the needle tip of the attached hypodermic injection needle. The needle filter captures particulate matter in medicine that is aspirated through the hollow injection needle into the syringe cavity while inhibiting fluid flow therethrough external the hollow needle. The needle cap's tapered profile facilitates more complete emptying of medicine within the corners of the medicine vial. Sealing contact of the filter with the hollow needle's tip inhibits medicine from flowing into the dead space of the needle cap, between the filter and the needle hub. Thus, tapered, filtering needle caps of the present disclosure waste less medicine by more efficient vial emptying and less dead space within the to-be-discarded needle cap. Medicine is aspirated from the vial, through a needle filter within the needle cap, directly through the injection needle of the syringe, while the needle cap is coupled to the syringe's needle hub. Upon completion of medicine aspiration into the syringe, the needle cap is removed, exposing the syringe's previously attached injection needle and the syringe is ready for administration of filtered medicine to the patient. By having the injection needle already attached to the syringe prior to medicine aspiration there is reduced likelihood of potential syringe contamination that might otherwise arise in a two-stage medicine administration process by first removing a used, filtered fill needle from the hub of the syringe and replacing it with a hypodermic injection needle.

One aspect of the present disclosure pertains to a medical syringe including a syringe barrel having proximal and distal ends and a syringe cavity therein. The syringe has a plunger and coupled plunger stopper in the proximal end of the syringe barrel. A male Luer connector is on the distal end of the syringe barrel, in fluid communication with the syringe cavity. A Luer needle hub is coupled to the male Luer connector, in fluid communication with the syringe cavity. A hollow syringe needle, having a proximal end, is coupled to the Luer needle hub, in fluid communication with the syringe cavity, and a distal open needle tip. The syringe is coupled to a needle cap having a tubular body with an inner wall surface defining a cap internal cavity therein. The needle cap captures the distal open needle tip. The inner wall surface of the needle cap further defines an open-ended, female Luer-taper profile, first cavity portion on a proximal end of the tubular body that is coupled to the needle hub, and an open cap tip on a distal end of the tubular body. A fluid-permeable needle filter is oriented within the cap internal cavity. The needle filter has a distal end facing the open cap tip and a proximal end in abutting, fluid-sealed contact with open needle tip. The needle filter divides the cap internal cavity into second proximal and third distal portions that are separated from each other. The needle filter captures particulate matter in fluid aspirated through the hollow needle into the syringe cavity while inhibiting fluid flow therethrough external the hollow needle from the third, distal portion of the hollow cavity into the second, proximal portion of the hollow cavity. The tubular body has a tapered, frusto-conical outer surface profile on the distal end thereof, which terminates in the open cap tip. In some embodiments, the proximal end of the needle filter defines a partial depth through-bore that does not communicate with the distal end of the filter; the needle tip is captured within and in sealing engagement with the filter through-bore. In other embodiments, the needle tip pierces and penetrates the proximal end of the needle filter but does not penetrate the distal end of the filter. In yet other embodiments, the needle tip abuts and compresses the proximal end of the needle filter.

Another aspect of the present disclosure pertains to a syringe including a syringe barrel having proximal and distal ends and a syringe cavity therein. A plunger and coupled plunger stopper are oriented in the proximal end of the syringe barrel. There is a male Luer connector on the distal end of the syringe barrel, in fluid communication with the syringe cavity. A Luer needle hub is coupled to the male Luer connector of the syringe barrel, in fluid communication with the syringe cavity. A hollow syringe needle has a proximal end coupled to the Luer needle hub, in fluid communication with the syringe cavity, and a distal open needle tip. The syringe includes a needle cap having a tubular body with an inner wall surface defining a cap internal cavity therein, which captures the distal open needle tip. The inner wall surface of the needle cap further defines an open-ended, female Luer-taper profile, first cavity portion on a proximal end of the tubular body that is coupled to the needle hub. The needle cap's inner wall surface defines an open cap tip on a distal end of the tubular body, a retention shoulder axially spaced from the open cap tip and a radially inwardly-directed lip axially spaced from the retention shoulder. A porous polymer construction, fluid-permeable needle filter is oriented within the cap internal cavity, interposed between the retention shoulder and radially inwardly-directed axial lip. The needle filter has a distal end facing the open cap tip and a proximal end defining a partial depth through-bore that does not communicate with the distal end of the filter. The needle tip is captured within and in sealing engagement with the filter through-bore. The needle filter divides the cap internal cavity into second proximal and third distal portions that are separated from each other. The needle filter captures particulate matter in fluid aspirated through the hollow needle into the syringe cavity while inhibiting fluid flow therethrough external the hollow needle from the third, distal portion, of the hollow cavity into the second, proximal portion of the hollow cavity. The needle cap's tubular body has a tapered, frusto-conical outer surface profile on the distal end thereof, which terminates in the open cap tip.

An additional aspect of the present disclosure pertains to a syringe including a syringe barrel having proximal and distal ends and a syringe cavity therein. A plunger and coupled plunger stopper are oriented in the proximal end of the syringe barrel. A male Luer connector is oriented on the distal end of the syringe barrel, in fluid communication with the syringe cavity. A Luer needle hub is coupled to the male Luer connector, in fluid communication with the syringe cavity. The syringe includes a hollow syringe needle having a proximal end coupled to the Luer needle hub, in fluid communication with the syringe cavity, and a distal open needle tip. The syringe further includes a needle cap having a tubular body with an inner wall surface defining a cap internal cavity therein, which captures the distal open needle tip. The inner wall surface of the needle cap further defines an open-ended, female Luer-taper profile, first cavity portion on a proximal end of the tubular body that is coupled to the needle hub. The inner wall surface of the needle cap also defines an open cap tip on a distal end of the tubular body, a retention shoulder axially spaced from the open cap tip and a radially inwardly-directed lip that is axially spaced from the retention shoulder. A porous polymer construction, fluid-permeable needle filter is oriented within the cap internal cavity, interposed between the retention shoulder and radially inwardly-directed axial lip. The needle filter has a distal end facing the open cap tip and a proximal end. In this embodiment, the needle tip pierces penetrates, and is in sealing engagement with the proximal end of the needle filter. The needle filter divides the cap internal cavity into second proximal and third distal portions that are separated from each other. The needle filter captures particulate matter in fluid aspirated through the hollow needle into the syringe cavity while inhibiting fluid flow therethrough external the hollow needle from the third, distal portion, of the hollow cavity into the second, proximal portion of the hollow cavity. In this embodiment, the tubular body has a tapered, frusto-conical outer surface profile on its distal end, which terminates in the open cap tip.

### BRIEF DESCRIPTION OF THE DRAWINGS

Exemplary embodiments of the disclosure are further described in the following detailed description in conjunction with the accompanying drawings, in which:

FIG. 1 is an isometric view of a medical syringe with a coupled needle cap and needle shield of the present disclosure;

FIG. 2 is an axial cross-sectional view of the medical syringe with a coupled needle cap and needle shield of FIG. 1;

FIG. 3 is an axial cross-sectional view of the coupled needle cap, syringe hypodermic needle and needle hub of FIG. 2;

FIG. 4 is an exploded view of the medical syringe, needle cap and needle shield of FIG. 1;

FIG. 5 is a view of the needle cap and syringe of FIG. 1 aspirating medicine through a septum of a medicine vial;

FIG. 6 is an alternate embodiment of a needle cap, syringe hypodermic needle and needle hub; and

FIG. 7 is another alternate embodiment of a needle cap, syringe hypodermic needle and needle hub.

To facilitate understanding, identical reference numerals have been used, where possible, to designate identical elements that are common to the figures. The figures are not drawn to scale.

### DETAILED DESCRIPTION

A medical syringe with a filtering needle cap facilitates aspiration of filtered medicine into the syringe, through the septum of a medicine vial. Medicine, potentially including contaminating debris, such as glass shards, is aspirated from the vial, directly through a needle filter within the needle cap that is in sealed, abutting contact with a tip of the syringe's injection needle, while the needle cap is coupled to the needle hub of the syringe. The proximal end of the needle cap is coupled to a needle hub of the syringe, with the tip of the syringe needle in sealing contact and engagement with a proximal end of the needle filter. In use, an open tip, tapered distal end of the needle cap is inserted through the septum of the medicine vial septum. Retraction of the syringe plunger aspirates medicine, by negative pressure, through the open tip of the needle cap, directly through the needle filter and the hollow syringe needle, into the syringe chamber. The needle filter separates and blocks contaminants in the medicine payload from entering the hollow needle and the syringe cavity. Sealing contact between the proximal end of the needle filter and the injection needle tip inhibits accumulation of excess medicine within dead space between the proximal end of the needle filter and the needle hub. In this way, filtered medicine that was not aspirated into the syringe chamber is not wasted. The tapered distal tip of the needle cap facilitates complete aspiration of medicine from the medicine vial, especially around the corners of the vial. Upon completion of medicine aspiration into the syringe, the needle cap is removed, and the syringe is ready for administration of medicine to the patient.

Before describing several exemplary embodiments of the disclosure, it is to be understood that the disclosure is not limited to the details of construction or process steps set forth in the following description. The disclosure is capable of other embodiments and of being practiced or being conducted in many ways.

In this disclosure, a convention is followed wherein the distal end of the device is the end closest to a patient and the proximal end of the device is the end away from the patient and closest to a practitioner.

With respect to terms used in this disclosure, the following definitions are provided. As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is generally associated with a flush or medication administration syringe and can interlock and connect to the female end located on the vascular access device (VAD) or on a hypodermic needle hub. The Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard luer connector increases from the open distal end to the proximal end. A female standard luer connector decreases from the open proximal end to the distal end. According to ISO 80369-7:2016, a male standard luer connector has an outer cross-sectional diameter measured 0.75 mm from the distal end of the tip of between 3.970 mm and 4.072 mm. The length of the male standard luer taper is between 7.500 mm to 10.500 mm. The outer cross-sectional diameter measured 7.500 mm from the distal end of the tip is between 4.376 mm and 4.476 mm. As used herein, the phrases “male standard luer connector” and “female standard luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually to implement various aspects of the embodiments of the present disclosure.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

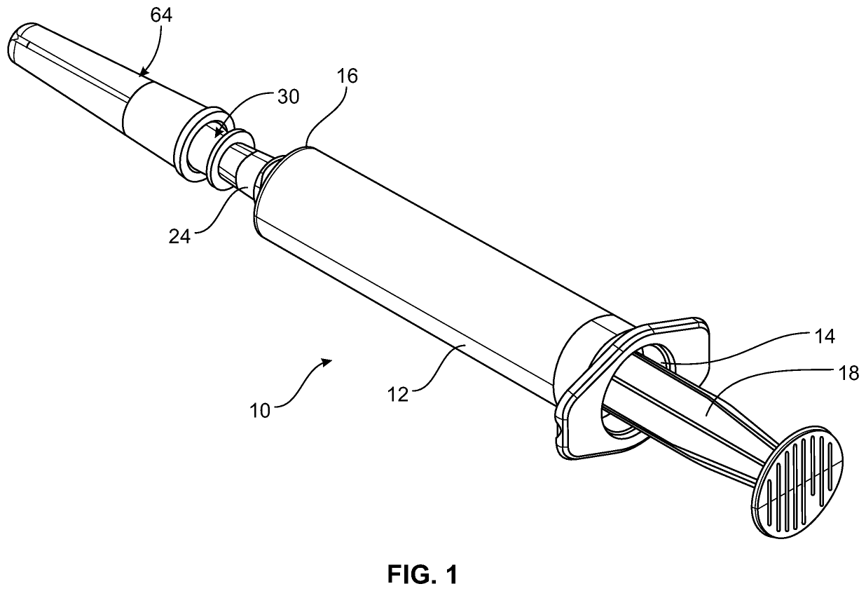

In an exemplary implementation of the embodiments of present disclosure, FIGS. 1-3 depict a syringe 10. The syringe 10 includes a syringe barrel 12, having proximal 14 and distal ends 16. A plunger 18 and a coupled plunger stopper 20 are inserted in the proximal end 14 of the syringe barrel 12. A syringe cavity 21 is defined between the distal end 16 of the syringe barrel 12 and a distal end of the plunger stopper 20. A male Luer connector 22 on the distal end 16 of the syringe barrel 12 is in fluid communication with the syringe cavity 21. A Luer needle hub 24 is coupled to the male Luer connector 22, which is also in fluid communication with the syringe cavity 21. A hollow syringe needle 26 has a proximal end coupled to the Luer needle hub 24 and a sharpened, distal open needle tip 28 for subcutaneous puncture of a patient. The distal open needle tip 28 is in fluid communication with the syringe cavity 21 via the lumen defined within the hollow needle 26.

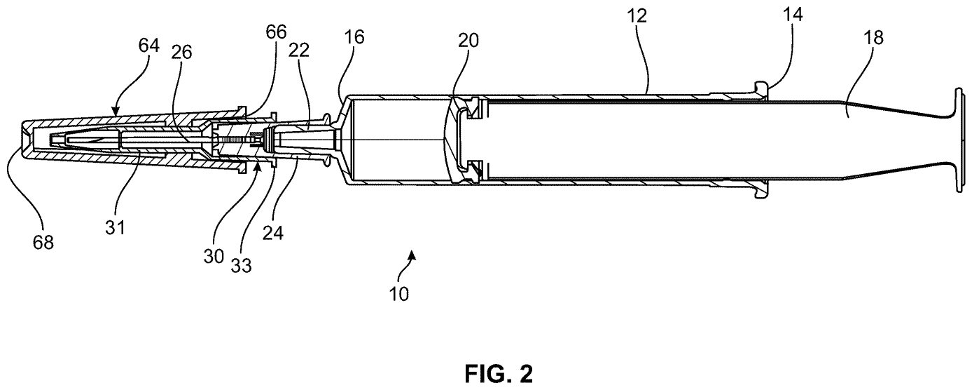

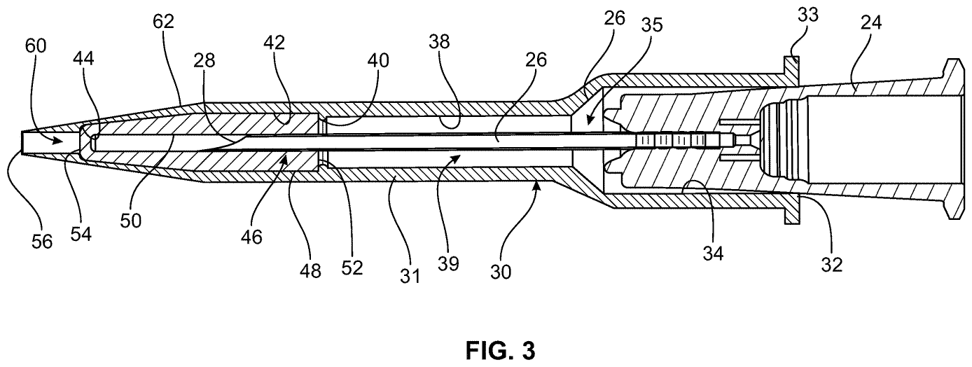

Referring to FIGS. 2 and 3, a needle cap 30 has a hollow tubular body 31, with a proximal end 32 that incorporates a radially outwardly projecting gripping flange 33. A first inner wall surface 34 of the hollow tubular body 31 defines an open-ended, female Luer-taper profile, first cavity portion 35. The first inner wall surface 34 is coupled to the exterior surface of the needle hub 24 in mutual slip-fit, friction engagement. In other embodiments, the first inner wall surface defines internal female threads of a threaded Luer connector that are engagement with corresponding male threads of a threaded Luer hub. The inner wall of the hollow tubular body 31 defines a necked portion 36 distal its first inner wall surface 34. The distal end of the necked portion 36 within the hollow tubular body 31 has a reduced diameter inner wall surface 38, which in turn defines a proximal second portion 39 of the needle cavity and an inwardly-projecting radial lip 40. The inner wall of the hollow tubular body 31 distal the radially projecting lip 40 further defines a filter cavity inner wall 42 and a filter retention shoulder 44.

A porous needle filter 46 is oriented within the filter cavity inner wall 42 between the inwardly-projecting radial lip 40 and the filter retention shoulder 44. Needle filter 46 has an outer diameter surface 48 in sealing engagement with the filter cavity inner wall 42. The needle filter 46 defines a partial through-bore 50 in its proximal axial end 52 that does not penetrate and/or communicate with its distal axial end 54. The partial through-bore 50 receives and captures the syringe needle 26 therein and has an inner diameter surface in sealing engagement with the outer surface of the syringe needle. The open needle tip 28 remains captured within the partial through-bore 50 of the needle filter 46 and is only in fluid communication with an open distal cap tip 56 of the needle cap 30 when fluid migrates through the porous filter material. In this way, the porous filter material blocks contaminants within the medicine payload from entering the open needle tip, the needle lumen and ultimately the syringe cavity 21. The needle filter 46 separates the internal cavity of the needle cap 30 into the proximal second portion 39, defined between its proximal axial end 52 and the first cavity portion 35 and a distal third portion 60 defined between its distal axial end 54 and the open distal cap tip 56 of the needle cap. In the syringe embodiment of FIGS. 1-4, the needle filter 46 is a separate component comprising a porous elastomeric material.

Potentially contaminated medicine payload aspirated through the open distal cap tip 56 only enters the distal third portion 60 of the needle cap 30 and flows through the distal end 54 of the needle filter 46. After the medicine has passed through filter 46 it is no longer contaminated; it is drawn through the open needle tip 28 and ultimately into the syringe chamber 12 of the syringe 10. The needle filter 46, in sealing engagement with filter cavity inner wall 42 and the outer diameter of the syringe needle 26, the blocks fluid leakage into the proximal second cavity portion 39 and the first cavity portion 35 of the needle cap 30, so that aspirated medicine is not trapped and wasted within the needle cap. In some embodiments, so-called “dead space” volume within the third distal cavity portion 60 of the needle cap 30 is minimized, in order to minimize wasted aspirated medicine. The filter 46 only draws medicine therethrough, from its proximal 52 to its distal 54 axial end under negative pressure during aspiration of the syringe 10, so medicine does not leak out of the syringe when there is no aspiration.

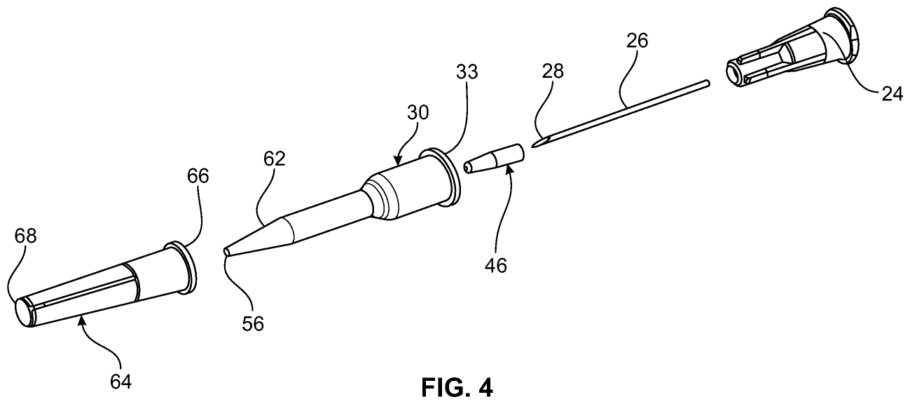

Referring to FIGS. 3 and 4, the outer exterior surface profile of the needle cap 30 forms a tapered, frusto-conical distal end 62, which terminates in the open distal cap tip 56. In other embodiments, the tapered distal end comprises other tapered polygonal profiles that terminate in the open distal cap tip.

A selectively removeable needle shield 64 is coupled to and covers the distal end of the needle cap 30 is shown in FIGS. 1, 2 and 4. The needle shield 64 has a proximal open end 66 and a blunt, closed tip 68 on its distal end. The needle shield 64 seals the open distal cap tip 56 to prevent entry of foreign matter into the needle cap 30 and the syringe 10; its blunt tip 68 also prevents inadvertent piercing of sterile syringe packaging that might otherwise occur if the tapered distal end 62 of the syringe was not covered by the needle shield.

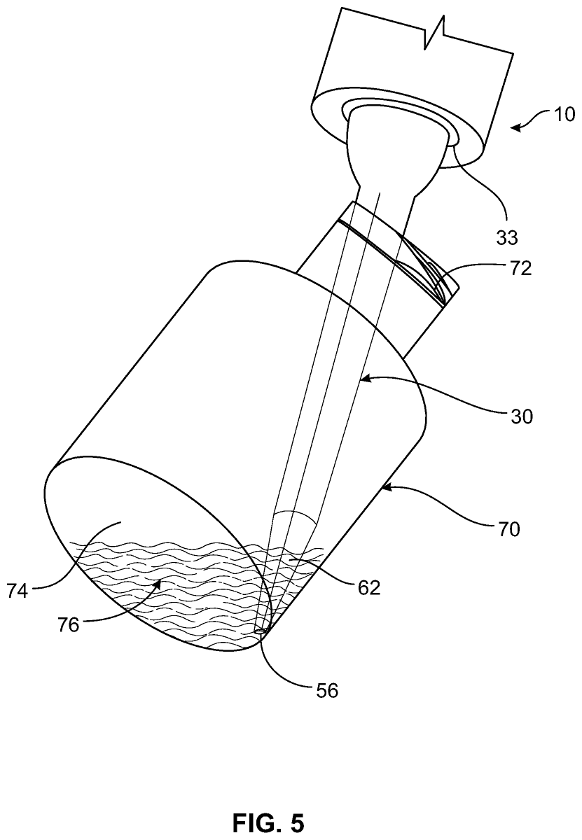

As shown in FIG. 5, the tapered distal end 62 of the tip of the needle cap 30 facilitates direct aspiration of medicine from a drug vial 70 while the syringe needle remains in situ within the needle cap. The tapered distal end 62 of the needle cap 30 pierces a septum 72 of the drug vial 70 and reaches the vial's bottom 74, to aspirate the medicine 76 or other medical liquid, such as saline flushing solution, directly into the syringe 10. The tapered distal end 62 of the needle cap 30 allows its open cap tip 56 to extract all medicine from corners of the vial bottom 74. The smooth, tapered distal end 62 of the needle cap 30 reduces potential infiltration of damaged vial septum 72 particulate matter into the vial that might otherwise contaminate the medicine 76 if a sharpened syringe needle tip were used directly to aspirate medicine from the vial 70. Any vial septum material as well as glass shards within the vial's medicine payload is filtered and blocked by needle filter 46. Beneficially, while the needle cap 30 covers the syringe needle, it also protects the syringe needle from bending or needle tip glinting contact damage. After aspirating the desired quantity of drug into the syringe chamber, a medical practitioner removes the needle cap 30 to expose the syringe's injection needle for subsequent injection into the patient. Handling of exposed, sharp injection needles is minimized because the needle and needle hub remain affixed to the syringe during medicine aspiration into the syringe.

In contrast, without the benefit of use of the filtering needle cap 30 of this disclosure, a medical practitioner exercising patient drug administration protocols would otherwise need to perform a two-stage process. As previously mentioned, the practitioner would first affix a blunt fill needle (with or without an internal filtering element) to a naked syringe Luer connector, by first removing its associated injection needle or by utilizing a syringe without an injection needle. After blunt fill needle affixation, the medical practitioner aspirates medicine from a vile into the syringe chamber. In the second stage, the practitioner thereafter replaces the blunt fill needle with an injection needle, in order to administer the drug to the patient. Use of a separate, known blunt fill needle, in the two-stage administration process, rather than a single-stage process with the syringe 10 with its needle cap 30 of the present disclosure, requires additional syringe preparation steps, thereby increasing possibility of administration error and potential sterility compromise.

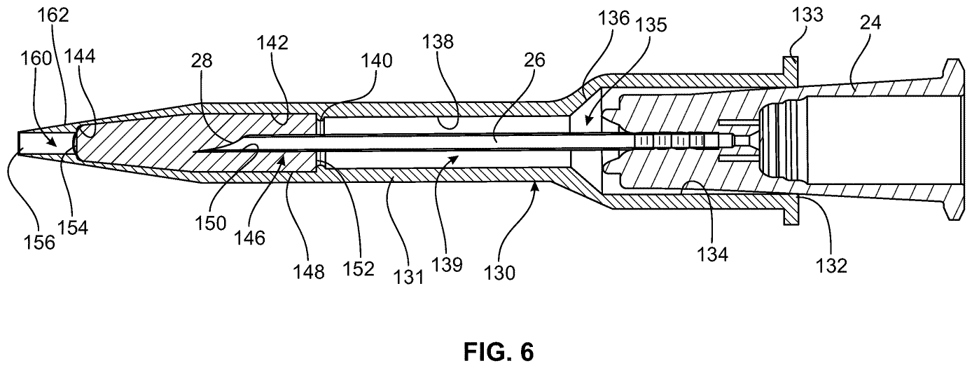

FIG. 6 depicts another embodiment of a needle cap of the present disclosure. The needle cap 130 is coupled to a syringe hub 24, its hypodermic needle 26 and sharpened needle tip 28. The needle cap 130 has a hollow tubular body 131, with a proximal end 132 that incorporates a radially outwardly projecting gripping flange 133. A first inner wall surface 134 of the hollow tubular body 131 defines an open-ended, female Luer-taper profile, first cavity portion 135. The first inner wall surface 134 is coupled to the exterior surface of the needle hub 24 in mutual slip-fit, friction engagement. In other embodiments, the first inner wall surface defines internal female threads of a threaded Luer connector that are engagement with corresponding male threads of a threaded Luer hub. The inner wall of the hollow tubular body 131 defines a necked portion 136 distal its first inner wall surface 134. The distal end of the necked portion 136 within the hollow tubular body 131 has a reduced diameter inner wall surface 138, which in turn defines a proximal second portion 139 of the needle cavity and an inwardly projecting radial lip 140. The inner wall of the hollow tubular body 131 distal the radially projecting lip 140 further defines a filter cavity inner wall 142 and a filter retention shoulder 144.

A porous needle filter 146 is oriented within the filter cavity inner wall 142 between the inwardly projecting radial lip 140 and the filter retention shoulder 144. Needle filter 146 has an outer diameter surface 148 in sealing engagement with the filter cavity inner wall 142. The sharpened needle tip 28 pierces the interior 150 of the needle filter 146 at the latter's proximal axial end 152, but the tip does not pierce the filter's distal axial end 154. The filter interior portion 150 receives and captures the sharpened tip 28 of the syringe needle 26 therein and is in sealing engagement with the outer surface of the syringe needle. The open needle tip 28 remains captured within the interior 150 of the needle filter 146 and is only in fluid communication with an open distal cap tip 156 of the needle cap 130 when fluid migrates through the porous filter material. In this way, the porous filter material blocks contaminants within the medicine payload from entering the open needle tip, the needle lumen and ultimately the syringe cavity 21. The needle filter 146 separates the internal cavity of the needle cap 130 into the proximal second portion 139, defined between its proximal axial end 152 and the first cavity portion 135 and a distal third portion 160 defined between its distal axial end 154 and the open distal cap tip 156 of the needle cap. In the syringe embodiment of FIG. 6, the needle filter 146 is a separate component comprising a porous elastomeric material.

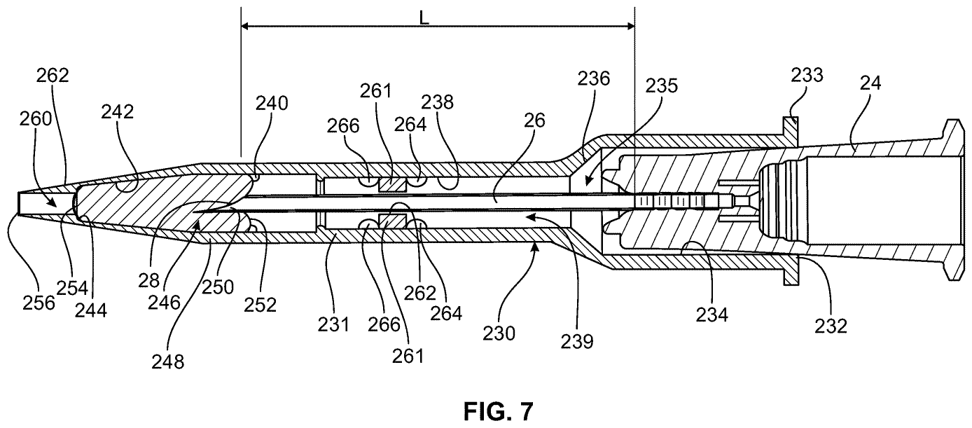

FIG. 7 depicts another embodiment of a needle cap of the present disclosure. The needle cap 230 is coupled to a syringe hub 24, its hypodermic needle 26 and sharpened needle tip 28. The needle cap 230 has a hollow tubular body 231, with a proximal end 232 that incorporates a radially outwardly projecting gripping flange 233. A first inner wall surface 234 of the hollow tubular body 231 defines an open-ended, female Luer-taper profile, first cavity portion 235. The first inner wall surface 234 is coupled to the exterior surface of the needle hub 24 in mutual slip-fit, friction engagement. In other embodiments, the first inner wall surface defines internal female threads of a threaded Luer connector that are engagement with corresponding male threads of a threaded Luer hub. The inner wall of the hollow tubular body 231 defines a necked portion 236 distal its first inner wall surface 234. The distal end of the necked portion 236 within the hollow tubular body 231 has a reduced diameter inner wall surface 238, which in turn defines a proximal second portion 239 of the needle cavity and an inwardly projecting radial lip 240. The inner wall of the hollow tubular body 231 distal the radially projecting lip 240 further defines a filter cavity inner wall 242 and a filter retention shoulder 244.

A porous needle filter 246 is oriented within the filter cavity inner wall 242 between the inwardly projecting radial lip 240 and the filter retention shoulder 244. Needle filter 246 has an outer diameter surface 248 in sealing engagement with the filter cavity inner wall 242. The sharpened needle tip 28 abuts and biases against the needle filter 246, forming a depression 150 at the latter's proximal axial end 252, but the tip does not pierce the filter's distal axial end 254. The proximal axial end 252 of the needle filter 246, at its depression 250, receives and captures the sharpened tip 28 of the syringe needle 26 there against and is in sealing engagement with the outer surface of the syringe needle. The open needle tip 28 remains in biased, sealing engagement against the proximal axial end 252 of the needle filter 246 and is only in fluid communication with an open distal cap tip 256 of the needle cap 230 when fluid migrates through the porous filter material. In this way, the porous filter material blocks contaminants within the medicine payload from entering the open needle tip, the needle lumen and ultimately the syringe cavity 21. The needle filter 246 separates the internal cavity of the needle cap 230 into the proximal second portion 239, defined between its proximal axial end 252 and the first cavity portion 235 and a distal third portion 260 defined between its distal axial end 254 and the open distal cap tip 256 of the needle cap. In the syringe embodiment of FIG. 7, the needle filter 246 is a separate component comprising a porous elastomeric material.

The needle cap 230 incorporates a needle support ring 261 to inhibit potential buckling or radial biasing of the needle 26 when it is in abutting contact with the proximal end 252 of the needle filter 248. The needle support ring 261 is selectively oriented axially within the needle cap 230 along the length L of the needle 26. The needle support ring 261 defines an inner annular surface 262 in spaced orientation from the outer circumferential surface of the needle 26. In other embodiments, the inner annular surface 262 is in sealing engagement with the outer circumferential surface of the needle 26, forming a secondary seal to prevent migration of medicine into the proximal second portion 239 of the needle cavity. The needle support ring 261 is retained within the needle cap 230 by first 264 and second 266 radial lips. In other embodiments, the needle support ring is integrally formed within the needle cap 230.

The syringe and syringe needle cap embodiments disclosed herein are constructed from medical grade materials known to one skilled in the art. In some embodiments, described barrels, plungers and shafts are fabricated with polypropylene, polyethylene, or polycarbonate polymers. In some embodiments, stoppers are fabricated with polyisoprene polymers or other known elastomers.

In some embodiments, the needle filters 46, 146, 246 are formed as cylinders that are press fitted into its corresponding needle cap, in radial compression with inner walls of needle cap. In other embodiments, such as the needle filter 46 of FIG. 4, the filter is preformed into a desired profile. In some embodiments, the needle filter is constructed of porous polymers, such as an elastomer of 30-70 Shore A hardness, with filtering efficacy optimized to filter out particles of size>5 μm. In some embodiments, the needle filter is fabricated by blowing out air or blowing agent during injection or hot press molding (such as microcellular injection molding). In other embodiments, the needle filter is fabricated by preferential solubilization or precipitation of mixed particles such as salt. In yet other embodiments, the needle filter is fabricated by thermal, laser or ultrasonic welding or sintering of polymeric material. Exemplary polymeric materials include but are not limited to polypropylene, polyethylene, and polystyrene) particles.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A syringe comprising:
a syringe barrel having proximal and distal ends and a syringe cavity therein;
a plunger and coupled plunger stopper in the proximal end of the syringe barrel;
a male Luer connector on the distal end of the syringe barrel, in fluid communication with the syringe cavity;
a Luer needle hub coupled to the male Luer connector, in fluid communication with the syringe cavity;
a hollow syringe needle having a proximal end coupled to the Luer needle hub, in fluid communication with the syringe cavity, and a distal open needle tip;
a needle cap having a tubular body with an inner wall surface defining a cap internal cavity therein, which captures the distal open needle tip, the inner wall surface further defining an open-ended, female Luer-taper profile, first cavity portion on a proximal end of the tubular body that is coupled to the needle hub, and the inner wall surface defining an open cap tip on a distal end of the tubular body;
a fluid-permeable needle filter within the cap internal cavity, having a distal end facing the open cap tip and a proximal end in abutting, fluid-sealed contact with open needle tip, the needle filter dividing the cap internal cavity into second proximal and third distal portions that are separated from each other, the needle filter capturing particulate matter in fluid aspirated through the hollow needle into the syringe cavity while inhibiting fluid flow therethrough external the hollow needle from the third, distal portion, of the hollow cavity into the second, proximal portion of the hollow cavity; and
the tubular body having a tapered, frusto-conical outer surface profile on the distal end thereof, which terminates in the open cap tip.

2. The syringe of claim 1, further comprising the proximal end of the needle filter defining a partial depth through-bore that does not communicate with the distal end of the filter, the needle tip captured within and in sealing engagement with the filter through-bore.

3. The syringe of claim 1, further comprising the needle tip piercing and penetrating the proximal end of the needle filter, but not penetrating the distal end thereof.

4. Th syringe of claim 1, further comprising the needle tip abutting and compressing the proximal end of the needle filter, in sealing engagement with the proximal end.

5. The syringe of claim 1, the internal wall surface of the tubular body defining radially inwardly directed lip abutting proximal end of the needle filter and a retention shoulder abutting the distal end of the needle filter.

6. The syringe of claim 1, the tubular body defining a reduced diameter, necked portion intermediate the female Luer-taper profile, cavity portion and the tapered, frusto-conical distal end thereof, the needle filter oriented distal said necked portion.

7. The syringe of claim 1, the proximal end of the tubular body defining an outwardly projecting gripping flange.

8. The syringe of claim 1, the inner wall surface defining the first cavity portion of the cap internal cavity further comprising a female, Luer slip connector, mating with a corresponding male, Luer slip connector of the needle hub.

9. The syringe of claim 1, the inner wall surface defining the first cavity portion of the cap internal cavity further comprising a female, threaded Luer connector, mating with a corresponding male, threaded Luer connector of the needle hub.

10. The syringe of claim 1, further comprising a needle shield coupled to the distal end of the tubular body of the needle cap and sealing the open tip of the cap.

11. The syringe of claim 1, the needle filter capable of filtering out particles greater than or equal to 5 μm in size, which are entrained in fluid aspirated within the needle cap.

12. The syringe of claim 11, the needle filter radially compressed within the inner wall surface of the needle cap.

13. The syringe of claim 12, the needle filter comprising porous polymer made by the process selected from the group consisting of injection molding with a blowing agent or by sintering.

14. The syringe of claim 11, the needle filter comprising porous polymer made by the process selected from the group consisting of injection molding with a blowing agent or by sintering.

15. The syringe of claim 1, the needle filter comprising porous polymer made by the process selected from the group consisting of injection molding with a blowing agent or by sintering.

16. The syringe of claim 15, the needle filter comprising an elastomer of 30-70 Shore A hardness that is capable of filtering out particles greater than or equal to 5 μm in size, which are entrained in fluid aspirated within the needle cap.

17. The syringe of claim 1, further comprising a needle support ring coupled to the inner wall surface of the needle cap, oriented axially within the needle cap along the length of the needle, the needle support ring defining an inner annular surface in radially opposed orientation with an outer circumferential surface of the needle.

18. The syringe of claim 17, the inner annular surface of the needle support ring in sealing engagement with the outer circumferential surface of the needle.

19. A syringe comprising:
a syringe barrel having proximal and distal ends and a syringe cavity therein;
a plunger and coupled plunger stopper in the proximal end of the syringe barrel;
a male Luer connector on the distal end of the syringe barrel, in fluid communication with the syringe cavity;
a Luer needle hub coupled to the male Luer connector, in fluid communication with the syringe cavity;
a hollow syringe needle having a proximal end coupled to the Luer needle hub, in fluid communication with the syringe cavity, and a distal open needle tip;
a needle cap having a tubular body with an inner wall surface defining a cap internal cavity therein, which captures the distal open needle tip, the inner wall surface further defining an open-ended, female Luer-taper profile, first cavity portion on a proximal end of the tubular body that is coupled to the needle hub, and the inner wall surface defining an open cap tip on a distal end of the tubular body, a retention shoulder axially spaced from the open cap tip and a radially inwardly-directed lip axially spaced from the retention shoulder;
a porous polymer construction, fluid-permeable needle filter within the cap internal cavity, interposed between the retention shoulder and radially inwardly-directed axial lip, having a distal end facing the open cap tip and a proximal end defining a partial depth through-bore that does not communicate with the distal end of the filter, the needle tip captured within and in sealing engagement with the filter through-bore, the needle filter dividing the cap internal cavity into second proximal and third distal portions that are separated from each other, the needle filter capturing particulate matter in fluid aspirated through the hollow needle into the syringe cavity while inhibiting fluid flow therethrough external the hollow needle from the third, distal portion, of the hollow cavity into the second, proximal portion of the hollow cavity; and
the tubular body having a tapered, frusto-conical outer surface profile on the distal end thereof, which terminates in the open cap tip.

20. A syringe comprising:
a syringe barrel having proximal and distal ends and a syringe cavity therein;
a plunger and coupled plunger stopper in the proximal end of the syringe barrel;
a male Luer connector on the distal end of the syringe barrel, in fluid communication with the syringe cavity;
a Luer needle hub coupled to the male Luer connector, in fluid communication with the syringe cavity;
a hollow syringe needle having a proximal end coupled to the Luer needle hub, in fluid communication with the syringe cavity, and a distal open needle tip;
a needle cap having a tubular body with an inner wall surface defining a cap internal cavity therein, which captures the distal open needle tip, the inner wall surface further defining an open-ended, female Luer-taper profile, first cavity portion on a proximal end of the tubular body that is coupled to the needle hub, and the inner wall surface defining an open cap tip on a distal end of the tubular body, a retention shoulder axially spaced from the open cap tip and a radially inwardly-directed lip axially spaced from the retention shoulder;
a porous polymer construction, fluid-permeable needle filter within the cap internal cavity, interposed between the retention shoulder and radially inwardly-directed axial lip, having a distal end facing the open cap tip and a proximal end, the needle tip piercing, penetrating, and in sealing engagement with the proximal end of the needle filter, the needle filter dividing the cap internal cavity into second proximal and third distal portions that are separated from each other, the needle filter capturing particulate matter in fluid aspirated through the hollow needle into the syringe cavity while inhibiting fluid flow therethrough external the hollow needle from the third, distal portion, of the hollow cavity into the second, proximal portion of the hollow cavity; and
the tubular body having a tapered, frusto-conical outer surface profile on the distal end thereof, which terminates in the open cap tip.
